(19) United States
(12) Patent Application Publication
(10) Pub. No.: US 2025/0281734 A1
(43) Pub. Date: Sep. 11, 2025

(54) SURFACE MODIFICATION OF MEDICAL DEVICES

(71) Applicant: ABIOMED, Inc., Danvers, MA (US)

(72) Inventors: Vladimir GILMAN, Danvers, MA (US); Scott CORBETT, Danvers, MA (US)

(73) Assignee: ABIOMED, Inc., Danvers, MA (US)

(21) Appl. No.: 19/073,361

(22) Filed: Mar. 7, 2025

Related U.S. Application Data
(60) Provisional application No. 63/563,059, filed on Mar. 8, 2024.

(51) Int. Cl. A61M 60/80

(52) U.S. Cl. CPC A61M 60/80; A61M 2205/02

(57) ABSTRACT

Disclosed are techniques for delaying initiation of coagulation and suppressing fibrin formation. The disclosed techniques may include providing a percutaneous blood pump that may include a metal or ceramic surface (such as a surface of a shaft, bearing, rotor, stator, etc.) that has been modified with a bifunctional modifier. The modified surface may be within a motor section and/or pump section of the blood pump. The modified surface may be configured to interact with blood. When blood is allowed to interact with the modified surface, a desirable microenvironment may be formed. The bifunctional modifier may be a functionalized aminosilane, a functionalized aminosiloxane, and/or a functionalized silanetriol. The blood pump may be configured to have a purge fluid pass through at least a portion of the motor section and/or the pump section. The purge fluid may be free of anticoagulants.

### CROSS-REFERENCE TO RELATED APPLICATIONS

The present application claims priority to U.S. Provisional Patent Application No. 63/563,059, filed Mar. 8, 2024, the contents of which are incorporated by reference herein in its entirety.

### TECHNICAL FIELD

The present disclosure relates to medical devices, such as blood pumps (e.g., an intravascular blood pump, to support a blood flow in a patient's blood vessel and methods for purging such a pump in operation while inserted into a patient) having a blood-contacting surface.

### BACKGROUND

Blood pumps of different types are known, such as axial blood pumps, centrifugal blood pumps, or mixed-type blood pumps, where the blood flow is caused by both axial and radial forces. One example of a blood pump is the IMPELLA® line of blood pumps (e.g., IMPELLA 2.5® blood pump, IMPELLA CP® blood pump, IMPELLA 5.5® blood pump, etc.) which are products of Abiomed of Danvers, Mass. Intravascular blood pumps may be inserted into a patient's vessel such as the aorta by means of a catheter.

In some pump designs, a purge fluid may be deployed to keep blood from entering the mechanism and to mitigate the effects of blood on the pump mechanisms. The purge fluid may include aqueous dextrose solution (e.g., D5). In some instances, such a purge fluid also may include an anticoagulant such as heparin (typically the sodium salt of heparin), which is thought to keep the blood from coagulating in the gap between pump components such as an impeller shaft and the housing. In other instances, sodium bicarbonate has been introduced as an alternate to heparin.

### BRIEF DESCRIPTION OF DRAWINGS

Hereinafter, the invention will be explained by way of example with reference to the accompanying drawings. The accompanying drawings are not intended to be drawn to scale. In the drawings, each identical or nearly identical component that is illustrated in various figures is represented by a like numeral. For purposes of clarity, not every component may be labelled in every drawing.

FIG. 1 shows a schematic illustration of a first surface modifier according to one embodiment.

FIG. 2 shows an illustration of a portion of a blood pump, where a distal end is disposed within a right ventricle of a heart.

FIG. 3 shows an illustration of a blood pump according to one embodiment.

FIG. 4 shows an illustration of a blood pump according to another embodiment.

FIG. 5 is an illustration of a system that includes a blood pump.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

As is known, blood pumps may be deployed in patients that require critical and life-saving care. Blood pumps also may be used to support high-risk procedures. In some pump designs, a purge fluid may be deployed to keep blood from entering the pump mechanism and to mitigate the effects of blood on the pump mechanisms. For example, the purge fluid may include an aqueous dextrose solution (e.g., D5), such as a 5% dextrose in water solution. In some instances, the purge fluid may also include an anticoagulant such as heparin (e.g., the sodium salt of heparin), which is thought to keep the blood from coagulating in the gap between pump components such as an impeller shaft and the housing. As will be appreciated, there may be instances in which heparin may not be suitable for all patients. In such instances, an alternative to heparin, such as sodium bicarbonate, has been appreciated by the inventors.

The inventors have also recognized that a sodium bicarbonate purge fluid may provide advantageous environmental conditions that may delay initiation of coagulation and suppress fibrin formation. For example, an overall decreased rate of clot formation and/or a reduction in the probability of protein denaturation and platelet activation due to an increased pH may be achieved. The inventors have also recognized that a similar advantageous microenvironment may be created by surface modifications (e.g., pH surface modifications) of blood contacting surfaces. For example, as disclosed herein, advantages may be achieved by surfacing modifying plastic and/or elastomeric surfaces, such as thermoplastic polyurethanes. In some embodiments, for polyurethane and/or pVAX (copolyoxalate containing vanillyl alcohol (VA)) surfaces, a carbonade, aldehyde, and/or amine first modifier may be used. A carboxyl group also may be used as a modifier.

As disclosed herein, the inventors recognized the benefit of providing a percutaneous blood pump that includes a surface modified plastic and/or elastomeric surface). As will be appreciated, the surface modification may be applied to a cannula portion, a catheter portion, or another suitable portion of the blood pump having a suitable plastic and/or elastomeric surface.

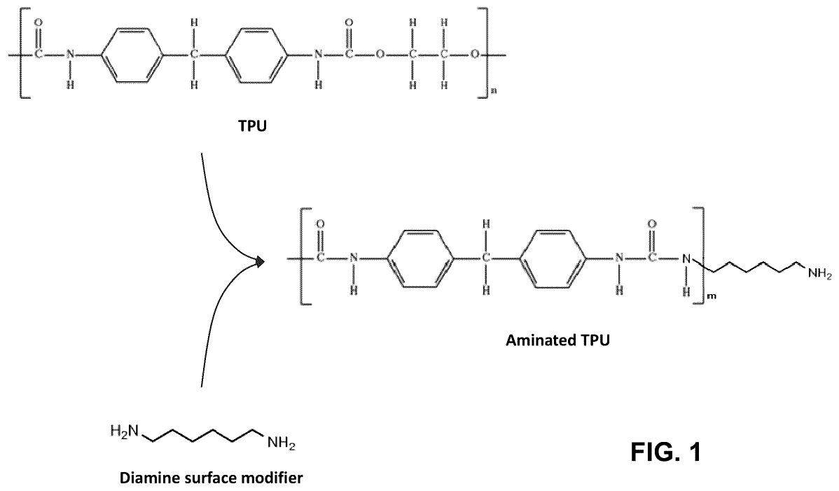

A schematic for forming the modified surface, such as TPUs, can be seen in FIG. 1. For example, as shown in FIG. 1, the addition of a diamine to the TPU may be achieved via a diamine aminolysis. As a result of this reaction, polymer chain scission in a thin molecular layer of TPU may occur with a simultaneous replacement of polyol residue in the TPU structure by diamine. The remainder primary amino group of diamine molecule may thereafter serve as a permanent surface pH modifier. The TPU may be any appropriate TPU used for medical purposes. The diamine may be any appropriate diamine. The diamine may be a primary diamine.

In some embodiments, the diamine may be a linear diamine. The linear diamine may have a carbon chain between 2 and 10 carbons in length, such as between 3 and 8 carbons in length. The linear diamine may have a carbon chain of at least 2, 3, 4, or 5 carbons, and no more than 6, 7, 8, 9, or 10 carbons in length. Non-limiting examples include pentane-1,5-diamine, hexane-1,6-diamine, heptane-1,7-diamine, and octane-1,8-diamine.

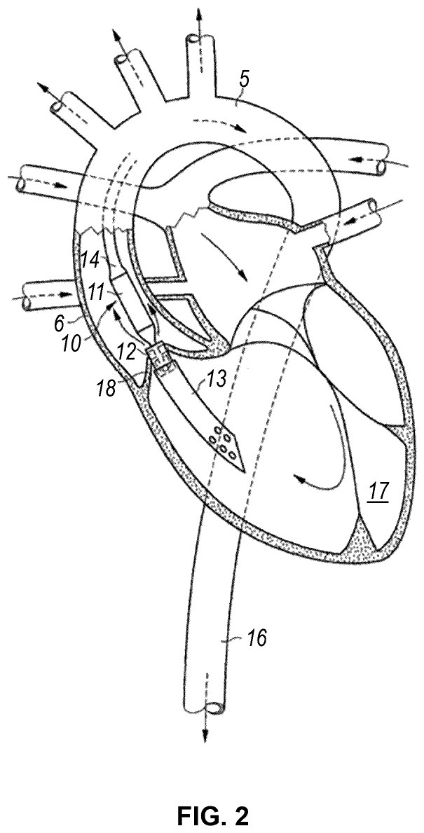

FIG. 2 illustrates the employment of a percutaneous blood pump for supporting, in this particular example, the left ventricle. As shown in this figure, the blood pump may include a catheter (14) and a pumping device (10) attached to the catheter (14). The pumping device (10) may include a motor section (11) and a pump section (12) which are disposed coaxially one behind the other and result in a rod-shaped construction form. The pump section (12) may include an extension in the form of a flexible suction hose (13), a tubular member often referred to as “cannula.” An impeller may be provided in the pump section (12) to cause blood flow from a blood flow inlet (20), through a portion of the pumping device, such as through a portion of the motor section and/or the pump section), to a blood flow outlet (21), and rotation of the impeller is caused by an electric motor disposed in the motor section (11). The blood pump may be placed such that it lies primarily in the ascending aorta (6) leading to the aortic arch (5). The aortic valve (18) comes to lie, in the closed state, against the outer side of the pump section 12 or its suction hose (13) that lies substantially in the left ventricle (17). The blood pump with the suction hose (13) in front may be advanced into the represented position by advancing the catheter (14), optionally employing a guide wire. In so doing, the suction hose (13) may pass the aortic valve (18) retrograde, so the blood is sucked in through the suction hose (13) and pumped into the aorta (16).

As will be appreciated, the use of the percutaneous blood pump need not be restricted to the application represented in FIG. 2, which merely involves a typical example of application. Thus, the pump can also be inserted through other peripheral vessels, such as the subclavian artery. Alternatively, reverse applications for the right ventricle may be envisioned. As will be further appreciated, other suitable pump arrangements may be used in other embodiments.

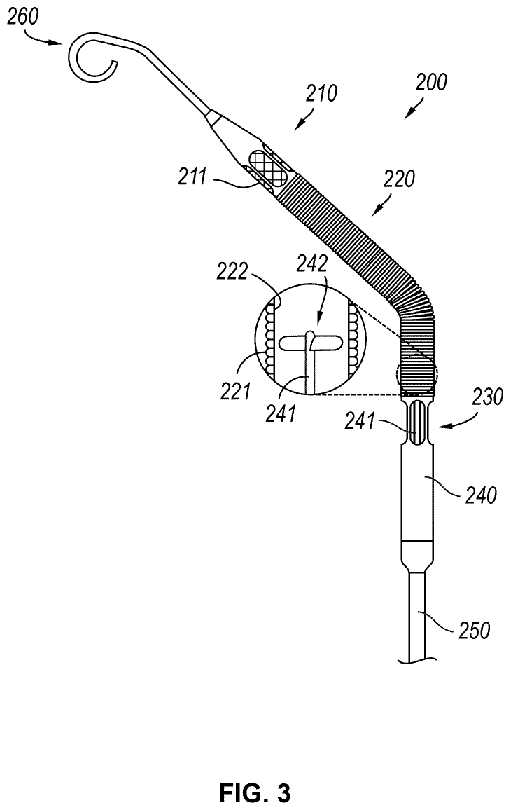

Referring to FIG. 3, a blood pump (200) can be seen, where any component comprising a plastic and/or elastomeric surface (such as a TPU or other suitable surface) may include a surface modified TPU as disclosed herein. The blood pump may include a cannula (220) operably coupled to a distal end of catheter (250). A flexible atraumatic tip (e.g., distal extension (260)) may be coupled to a distal end of the blood pump. The cannula may have an inner surface (222) and an outer surface (221). The outer surface may be exposed to, e.g., blood vessel walls and blood flowing around the blood pump. The inner surface may be expose to, e.g., blood flowing through the cannula. The cannula (220) may, optionally, include an impeller (242) disposed within the cannula (220). Optionally, the impeller may be within a housing (240). Housing (240) may include, e.g., the impeller and/or an electric motor. The housing (which may sometimes be referred to as a pump housing) may be disposed between the catheter and the cannula.

The motor may be coupled to the impeller via a drive shaft (241), which may be a rigid drive shaft. In some embodiments, when the impeller rotates, blood is configured to flow through an inlet (210), through the cannula, and out an outlet (230). The inlet may have an inflow cage (211) disposed over or at the inlet.

In some embodiment, the distal extension (260), the inflow cage (211), the cannula (220) (including, for example, the inner surface (222) and/or the outer surface (221)), and/or the catheter (250) may be composed of one or more embodiments of the surface modified TPU. In some embodiments, any surface modified TPU used in the blood pump has a single composition. In some examples, a first part of the blood pump (the cannula) may be configured with a surface-modified TPU having a first composition, and a second part of the blood pump (such as the distal extension) may be configured with a surface modified TPU having a second composition different from the first composition.

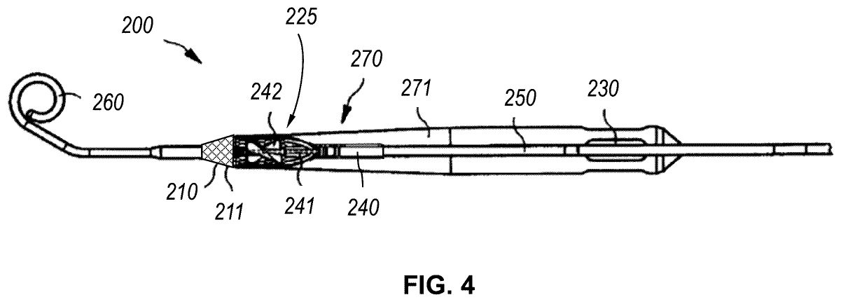

In FIG. 4, a different blood pump design can be seen. There, the blood pump (200) is shown as having an expandable pump housing (225) which may include one or more surface modified TPUs. An impeller (242) in the expandable pump housing may be configured to cause blood to flow through the inlet (210), through an intermediate portion of the expandable pump housing, out of the pump housing an into a volume of space (271) defined by an outflow tube (270). The outflow tube may include one or more surface modified TPUs. The blood then exits at the end of the outflow tube, through an outlet (230).

If purge fluids are utilized, purge flow rates may be in the range of about 2 mL/hour to about 30 mL/hour. This results in a purge pressure of about 1100 mmHg to about 300 mmHg. Typical purge flows for some blood pumps are about 5 mL/hour to about 20 mL/hour, although specific models may have purge flows from about 2 mL/hour to about 10 mL/hour.

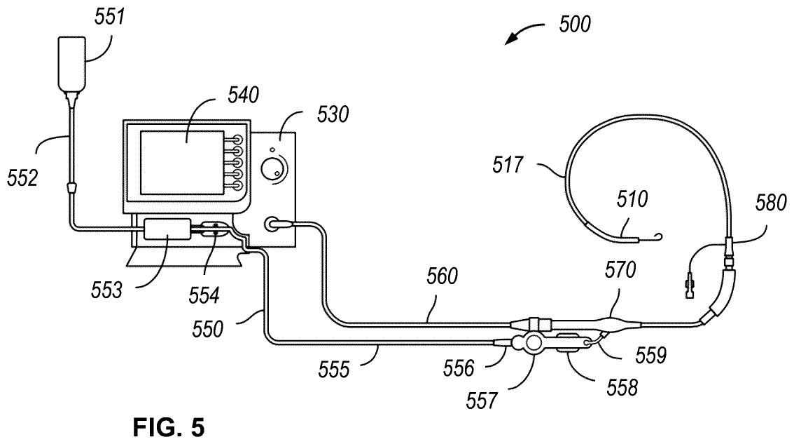

Referring to FIG. 5, a blood pump assembly (500) may include a blood pump (510) fluidically connected to a container (551) (such as a purge bag) that contains a purging fluid as disclosed herein, through a purging device (553). The blood pump assembly (500) also may include a controller (530) (e.g., an AUTOMATED IMPELLA CONTROLLER® (AIC) blood pump controller from Abiomed, Inc., Danvers, MA), a display (540), a connector cable (560), a plug (570), and a repositioning unit (580). As shown, the controller (530) may include a display (540). Controller (530) may monitor and controls blood pump (510). During operation, purging device (553) may deliver a purge fluid as disclosed herein to blood pump (510) through a first line (550, 555) (e.g., a tube), through one or more components (556, 557, 558, 559) and through a catheter tube (517), such as to prevent blood from entering the motor (not shown) within a motor housing of the pump. Connector cable (560) may provide an electrical connection between blood pump (510) and controller (530). Plug (570) connects catheter tube (517), purging device (553), and connector cable (560). In some embodiments, plug (570) may include a memory for storing operating parameters in case the patient needs to be transferred to another controller. Repositioning unit (580) may be used to reposition blood pump (510). As shown in this view, the fluid line (550, 555) may be separate from the connector cable (560) having one or more electrical wires.

One or more of the TPUs in the percutaneous blood pump, such as one or more TPU surfaces that may contact blood (e.g., one or more surfaces of a cannula, catheter, etc.) may be surface modified as disclosed herein.

In some embodiments, a method may include operating the pump, which may include rotating the impeller of the pump. In that regard, the method may involve pumping a flow of blood from the blood flow inlet, through a portion of the pumping device, such as through a portion of the motor section and/or the pump section, and out a blood flow exit. In such embodiment, the blood may pass through one or more of the modified surfaces (e.g., through the axial gaps disclosed herein). As described herein, such modified surfaces may create microenvironments that may minimize and/or prevent agglomeration of protein as blood passes therethrough.

In various aspects, a method for creating a microenvironment for delaying initiation of coagulation may be provided. The method may include providing a percutaneous blood pump comprising a surface modified TPU as disclosed herein. This may include introducing the blood pump into a blood vessel of a patient. This may include operating the blood pump in such a manner that blood flowing through the blood pump interacts with the surface modified TPU.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive. It will further be understood that any reference herein to subject matter known in the field does not, unless the contrary indication appears, constitute an admission that such subject matter is commonly known by those skilled in the art to which the present technology relates.

What is claimed is:

1. A percutaneous blood pump comprising:
a pumping device coupled to a catheter, the pumping device comprising a motor section coupled to a pump section, the pump section configured to cause blood to flow from a blood inlet of the pumping device to a blood outlet of the pumping device,
wherein at least one surface of the percutaneous blood pump comprises a surface modified thermoplastic polyurethane (TPU).

2. The percutaneous blood pump of claim 1, wherein the surface modified TPU is an aminated TPU.

3. The percutaneous blood pump of claim 1, wherein the catheter comprises the surface modified TPU.

4. The percutaneous blood pump of claim 1, wherein a cannula of the pumping device comprises the surface modified TPU.

5. The percutaneous blood pump of claim 1, wherein the surface modified TPU comprises a first surface modified TPU at a first location and a second surface modified TPU at a second location different from the first location.

6. The percutaneous blood pump of claim 5, wherein the first surface modified TPU and the second surface modified TPU are identical.

7. The percutaneous blood pump of claim 5, wherein the first surface modified TPU and the second surface modified TPU are different.

8. The percutaneous blood pump of claim 1, wherein the surface modified TPU is formed from a linear diamine.

9. The percutaneous blood pump of claim 8, wherein the linear diamine comprises between 2 and 10 carbons.

10. A method for creating a microenvironment for delaying initiation of coagulation, comprising:
providing a percutaneous blood pump comprising a surface modified thermoplastic polyurethane (TPU).

11. The method of claim 10, wherein the surface modified TPU is configured to contact blood.

12. The method of claim 10, wherein the surface modified TPU is a surface of a catheter or cannula.

13. The method of claim 10, wherein the surface modified TPU is an aminated TPU.

14. The method of claim 10, wherein the surface modified TPU comprises a first surface modified TPU at a first location and a second surface modified TPU at a second location different from the first location.

15. The method of claim 14, wherein the first surface modified TPU and the second surface modified TPU are identical.

16. The method of claim 14, wherein the first surface modified TPU and the second surface modified TPU are different.

17. The method of claim 10, wherein the surface modified TPU is formed from a linear diamine.

18. The method of claim 17, wherein the linear diamine comprises between 2 and 10 carbons.
